(19) United States
(12) Patent Application Publication
(10) Pub. No.: US 2024/0252705 A1
(43) Pub. Date: Aug. 1, 2024

(54) ULTRAVIOLET STERILIZATION DEVICE AND OPERATION CONTROL METHOD THEREOF

(71) Applicant: ADVANCE SOLUTION CO., LTD, Gyeonggi-do (KR)

(72) Inventor: Doo Hyun CHAE, Gyeonggi-do (KR)

(21) Appl. No.: 18/564,683

(22) PCT Filed: Aug. 5, 2022
(86) PCT No.: PCT/KR2022/011676

§ 371 Date: Nov. 28, 2023

(30) Foreign Application Priority Data
Sep. 23, 2021 (KR) 10-2021-0125836

(51) Int. Cl. A61L 2/24; A61L 2/10; A61L 2/26

(52) U.S. Cl. CPC A61L 2/24; A61L 2/10; A61L 2/26; A61L 2202/11; A61L 2202/14; A61L 2202/25

(57) ABSTRACT

An ultraviolet sterilization device and an operation control method thereof are disclosed. The ultraviolet sterilization device comprises: an ultraviolet lamp which is mounted in a body part and generates ultraviolet rays; two reflectors which each have a surface reflecting ultraviolet rays and are mounted behind the ultraviolet lamp to be rotatable about rotational axes disposed adjacent to each other respectively; a sterilization angle adjustment unit which rotates each of the reflectors about the corresponding rotational axis; a sensor unit which includes a distance sensor for generating a distance measurement value of the distance to an object to be sterilized; and a control unit which controls the operation of the sterilization angle adjustment unit for rotating each of the two reflectors so as to form a predetermined sterilization angle corresponding to the distance measurement value.

### TECHNICAL FIELD

The present invention relates to ultraviolet sterilization device and method of controlling thereof.

### BACKGROUND

Ultraviolet ray has the property of having a strong sterilizing effect without generating ozone harmful to the human body, so ultraviolet sterilization devices using ultraviolet ray are widely used for various purposes.

In general, it is known that ultraviolet sterilization can sterilize up to about 99.9% of bacteria and viruses within 1 to 2 seconds, effectively eliminating almost all bacteria and viruses, and since not adding chemicals, preventing secondary contamination of the surrounding environment.

Ultraviolet sterilization devices are used for various purposes such as household, industrial, and medical purposes, but since ultraviolet ray is harmful on the human body, there is a risk of using ultraviolet sterilization devices in spaces where people frequently enter and exit, such as living rooms or elevators.

To eliminate this risk, a technology is being used to ensure safety by turning off an ultraviolet lamp when a sensor detects an entry of a person into a space being sterilized by the ultraviolet sterilization device.

However, there is a problem with stopping an operation of the ultraviolet sterilization device by detecting the presence of a person in a space where people frequently enter and exit, such that the sterilization does not proceed even though the space is continuously polluted by the entry and exit of people, and the contamination of the space increases.

### DETAILED DESCRIPTION

### Technical Objectives

The present invention is intended to provide an ultraviolet sterilization device and a method for controlling the operation thereof that can be universally utilized in various spaces (e.g., restaurants, operating rooms in hospitals, toilets in rest areas, elevators, etc.) as a sterilization power can be adaptively adjusted corresponding to the distance from a target surface.

The present invention is intended to provide an ultraviolet sterilization device and a method of operation thereof that can ensure safety even during uninterrupted sterilization processing by preemptively adjusting a range of sterilization region when a moving object (e.g., a person, etc.) is expected to enter the sterilization region.

The present invention is intended to provide an ultraviolet sterilization device and a method of controlling its operation that enable thorough sterilization processing of a target space by removing blind areas where ultraviolet rays cannot reach through posture adjustment, etc.

Other objects of the present invention may be easily understood through the following description.

### Technical Solution

According to one aspect of the present invention, there is provided an ultraviolet sterilization device including an ultraviolet lamp, mounted on an interior of a body, configured for generating ultraviolet rays, a pair of reflectors, having surfaces reflecting the ultraviolet rays, being rotatable around respective rotation axes located adjacent to each other at the rear of the ultraviolet lamp, a sterilization angle adjusting unit, configured for rotating each reflector around its corresponding rotation axis, a sensor unit, having a distance detection sensor that generates a distance measurement value related to the distance to a target surface and a control unit, configured for controlling an operation of the sterilization angle adjusting unit to rotate each of the pair of reflectors so that a predetermined sterilization angle corresponding to the distance measurement value is formed, wherein the sterilization angle is an angle formed between the pair of reflectors in a direction opposite the ultraviolet lamp made and a sterilization region through which the ultraviolet rays generated by the ultraviolet lamp pass is limited by the pair of reflectors that are rotated and positioned to form the sterilization angle.

The control unit may use Equation 1 to calculate an operation time t for generating the ultraviolet rays for a sterilization operation.

$$\\ \begin{matrix}t = α \times \frac{{L}^{2}}{p} \times β & < Equation 1 >\end{matrix}$$

where α is a predetermined basic time constant value, L is the distance measurement value generated by the distance detection sensor, p is a power rating of the ultraviolet sterilization device or the ultraviolet lamp, and μ is a predetermined conditional time constant value corresponding to a size of the sterilization angle.

The control unit may control the sterilization angle adjusting unit such that the sterilization angle of a relatively small angle is formed as the distance measurement value becomes relatively distant, wherein the relatively small conditional time constant value is preset to be determined for the sterilization angle of the relatively small angle.

The ultraviolet sterilization device may further include a wheel mounted on a lower portion of the body and rotated by a drive motor. In order to ensure that the sterilization operation is performed for the entire lateral direction of the ultraviolet sterilization device, the control unit repeats a series of steps of controlling the drive motor to rotate the ultraviolet sterilization device by a predetermined angle with a vertical rotation axis as a rotation center to face the second direction after completing the sterilization operation for the operation time calculated while the ultraviolet sterilization device is postured to face a first direction, and calculating the sterilization angle and the operation time for performing the sterilization operation while postured to face a second direction.

The sensor unit may include a plurality of distance detection sensors each mounted at a predetermined location on a circumferential surface of the body, wherein the control unit refers to whether the distance measurement values generated by the plurality of distance detection sensor change to recognize a presence of a moving object, a direction of move of the moving object and a speed of move of the moving object.

When recognizing that the moving object is approaching the sterilization region, the control unit may control the sterilization angle adjusting unit to rotate corresponding reflector in order to reduce an area of a currently set sterilization region in such a way that the entry of the moving object into the sterilization region is prevented.

The control unit may control the ultraviolet lamp to turn off when the moving object is recognized as approaching toward the sterilization region with the reflector rotated to form a predetermined minimum sterilization angle.

Or, each of the pair of reflectors may include a first sub-reflector coupled to the rotation axis, and a second sub-reflector coupled to the first sub-reflector by means of a rotating unit, wherein when the moving object is recognized as approaching towards the sterilization region, the control unit controls the rotating unit to rotate the corresponding second sub-reflector in order to reduce an area of a currently set sterilization region in such a way that the entry of the moving object into the sterilization region is prevented.

The ultraviolet sterilization device may further include a shielding member having a structure that folds and unfolds being disposed adjacent to the inner circumferential surface of the body. When the moving object is recognized as approaching towards the sterilization region, the control unit controls the shielding member to unfold in order to reduce an area of a currently set sterilization region in such a way that the entry of the moving object into the sterilization region is prevented.

A surface of the pair of reflectors may be coated with magnesium oxide of a predetermined thickness.

The control unit may determine the sterilization angle to be formed based on a distance measurement that is maintained for a predetermined period of time or longer.

According to one aspect of the present invention, there is provided a computer program for performing al method of controlling operation of a ultraviolet sterilization device stored on a computer readable medium, wherein the computer program causes a computer to execute following steps: designating a sterilization angle according to a predetermined setting reference information using a distance measurement value of the distance from the target surface, and determining an operation time t using Equation 1, controlling a sterilization angle adjusting unit so that each of a pair of reflectors is rotated to form the sterilization angle and turning an ultraviolet lamp during the determined operation time, wherein the sterilization angle is an angle formed between the pair of reflectors in a direction opposite the ultraviolet lamp made and a sterilization region through which the ultraviolet rays generated by the ultraviolet lamp pass is limited by the pair of reflectors that are rotated and positioned to form the sterilization angle,

$$\\ \begin{matrix}t = α \times \frac{{L}^{2}}{p} \times β & < Equation 1 >\end{matrix}$$

where α is a predetermined basic time constant value, L is the distance measurement value generated by the distance detection sensor, p is a power rating of the ultraviolet sterilization device or the ultraviolet lamp, and μ is a predetermined conditional time constant value corresponding to a size of the sterilization angle.

Other aspects, features and advantages in addition to those described above will become apparent from the following drawings, claims and detailed description of the invention.

### Effects of Invention

According to an embodiment of the present invention, a sterilizing power can be adaptively adjusted according to a distance from a target surface, so that it can be used universally in various spaces (e.g., restaurants, operating rooms in hospitals, restrooms in rest areas, elevators, etc.).

Additionally, if a moving object (for example, a person, etc.) is expected to enter a sterilization region, safety can be ensured even during uninterrupted sterilization treatment by preemptively adjusting the range of the sterilization region.

In addition, it has the effect of enabling thorough sterilization of the target space by removing blind areas where ultraviolet rays cannot reach through posture adjustments, etc.

The effects that can be obtained from the present invention are not limited to the effects mentioned above, and other effects not mentioned can be clearly understood by those skilled in the art from the description below.

### BRIEF DESCRIPTION OF ACCOMPANYING DRAWINGS

FIG. 1 is a schematic block diagram of an ultraviolet sterilization device according to one embodiment of the present invention;

FIG. 2 exemplarily illustrates the shape of an ultraviolet sterilization device according to one embodiment of the present invention;

FIG. 3 exemplarily illustrates operation modes of the ultraviolet sterilization device according to one embodiment;

FIG. 4 exemplarily illustrates a configuration for adjusting a sterilization angle according to another embodiment of the present invention;

FIG. 5 exemplarily illustrates operations of an ultraviolet sterilization device according to one embodiment of the present invention;

FIG. 6 exemplarily illustrates a method of adjusting an adaptive sterilization region of an ultraviolet sterilization device according to one embodiment of the present invention;

FIG. 7 exemplarily illustrates a configuration for adjusting adaptive sterilizing area of an ultraviolet sterilization device according to other embodiments of the present invention;

FIG. 8 exemplarily illustrates a sterilization operation in an omnidirectional option according to one embodiment of the present invention; and

FIG. 9 is a flowchart showing a method for controlling the operation of the ultraviolet sterilization device according to one embodiment of the present invention.

### EMBODIMENTS OF INVENTION

The present invention is subject to various modifications and may have many different embodiments, certain of which are illustrated in the drawings and further described in the detailed description. However, this is not intended to limit the invention to any particular embodiment and is to be understood to include all modifications, equivalents, or substitutions that fall within the scope of the spirits and scope of the invention.

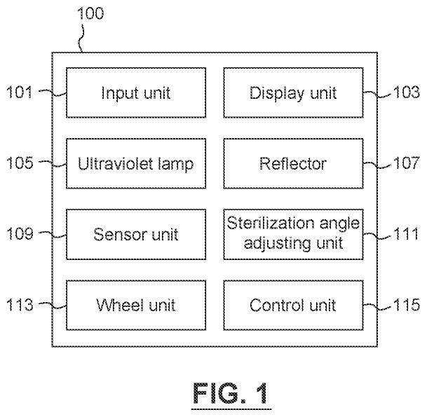

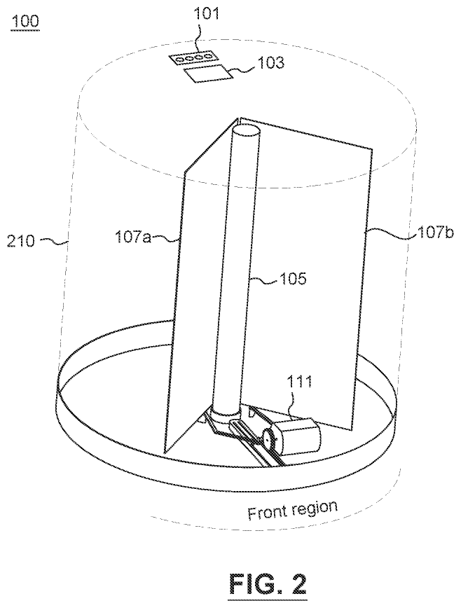

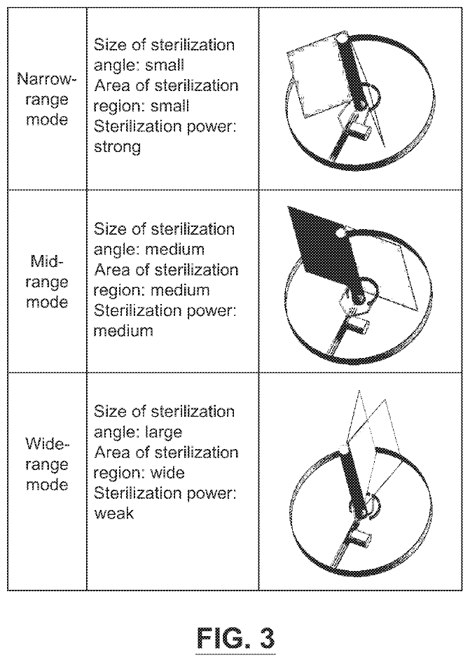

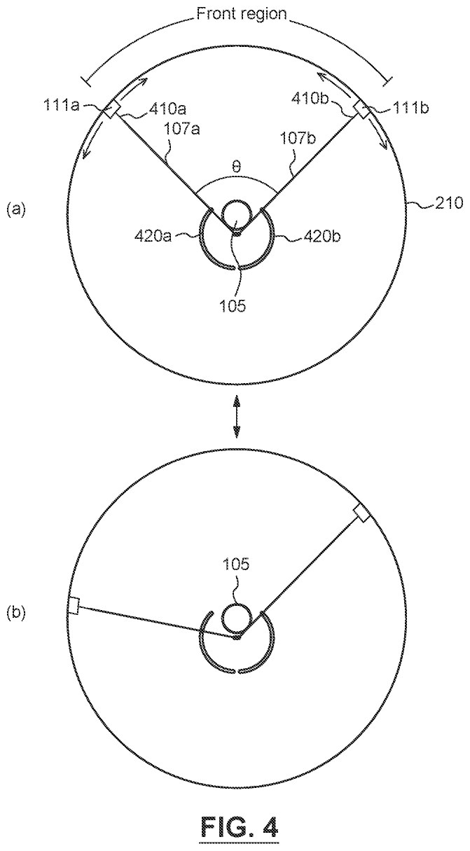

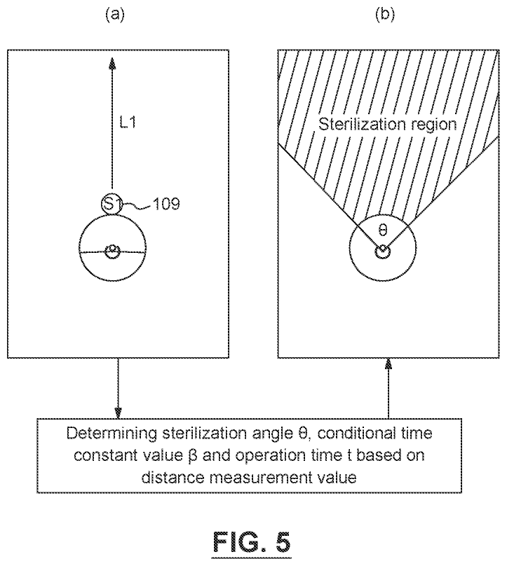

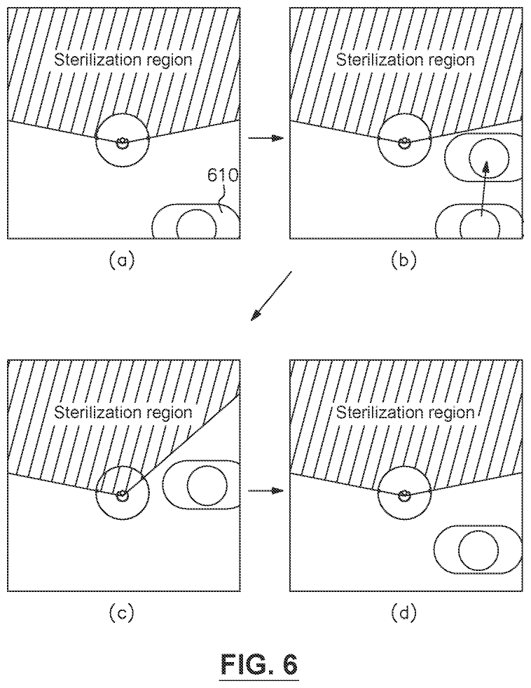

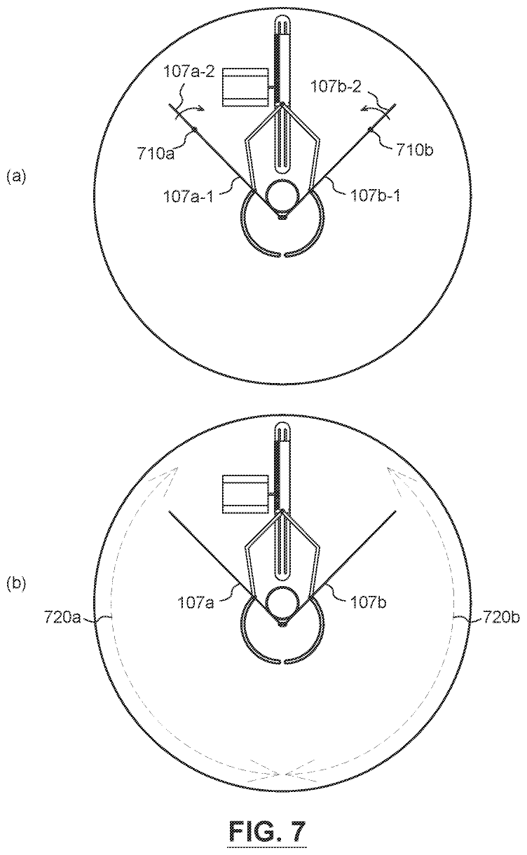

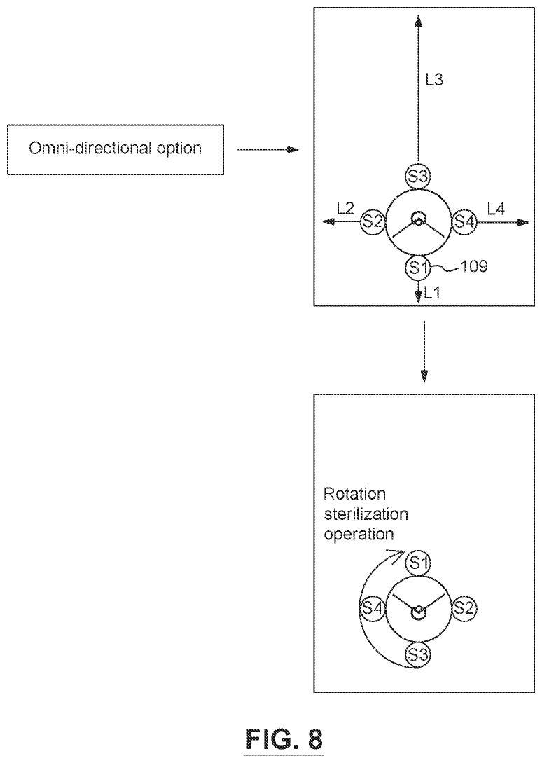

FIG. 1 is a schematic block diagram of an ultraviolet sterilization device according to one embodiment of the present invention, FIG. 2 exemplarily illustrates the shape of an ultraviolet sterilization device according to one embodiment of the present invention, and FIG. 3 exemplarily illustrates operation modes of the ultraviolet sterilization device according to one embodiment. FIG. 4 exemplarily illustrates a configuration for adjusting a sterilization angle according to another embodiment of the present invention, and FIG. 5 exemplarily illustrates operations of an ultraviolet sterilization device according to one embodiment of the present invention. FIG. 6 exemplarily illustrates a method of adjusting an adaptive sterilization region of an ultraviolet sterilization device according to one embodiment of the present invention, and FIG. 7 exemplarily illustrates a configuration for adjusting adaptive sterilizing area of an ultraviolet sterilization device according to other embodiments of the present invention, and FIG. 8 exemplarily illustrates a sterilization operation in an omnidirectional option according to one embodiment of the present invention.

Referring to FIG. 1, the ultraviolet sterilization device 100 may include an input unit 101, a display unit 103, an ultraviolet lamp 105, a reflector 107, a sensor unit 109, a sterilization angle adjusting unit 111, and a wheel unit 113 and a control unit115.

The input unit 101 is configured for receiving a user command for operating the ultraviolet sterilization device 100. The input unit 101 may be configured with, for example, one or more key buttons, and may also include a receiving means for the user to input the user command using a remote controller.

As an example, the user can use the input unit 101 to select an operation option to be sterilized by the ultraviolet sterilization device 100. Operation options may be various such as, for example, a manual option to perform the sterilization operation according to selection information specified by the user, an adaptive option to perform the sterilization operation corresponding to a distance to a target surface measured while the ultraviolet sterilization device 100 is placed, and an omni-directional option in which the device 100 adjust its posture in a preset manner (e.g., rotate 360 degrees) and performs the sterilization operation adaptively depending on the distance from a plurality of target surfaces.

The display unit 103 is configured for displaying an operating state of the ultraviolet sterilization device 100.

As illustrated in FIG. 2, the input unit 101 and the display unit 103 may each be disposed on one side of a body 210 of the ultraviolet sterilization device 100. The input unit 101 and the display unit 103 may be arranged independently, or may be integrated and implemented using a touch panel screen or the like.

The ultraviolet lamp 105 is configured for generating ultraviolet rays and is turned on or off under the control of the control unit 115. As illustrated in FIG. 2, the ultraviolet lamp 105 may be arranged in a vertically upright position in the internal space of the body 210.

While FIG. 2 illustrates a configuration where only one ultraviolet lamp 105 is disposed in an upright position perpendicular to the center point position of the body 210, as will be described later, it will be appreciated that two or more ultraviolet lamps 105 may be disposed spaced apart from each other, provided that they do not interfere with the rotation of the pair of reflectors 107a, 107b for adjustment of the sterilization region.

Ultraviolet rays can be divided into ultraviolet rays A (UV-A), ultraviolet B (UV-B), and ultraviolet C (UV-C) depending on the wavelength range, and the ultraviolet lamp 105 included in the ultraviolet sterilization device 100 may be one that emit ultraviolet rays, which belong to the wavelength range of ultraviolet C, having a wavelength range of about 100 nm to 280 nm and having a sterilizing power that can kill single-celled organisms (for example, contaminants such as viruses or bacteria).

The reflector 107 is configured for reflecting ultraviolet rays generated by the ultraviolet lamp 105, and its surface may be coated with magnesium oxide (MgO) of a predetermined thickness to increase reflectivity and thus improve sterilization effect.

As illustrated in FIGS. 2 to 4, the reflector 107 includes a pair of first reflector 107a and a second reflector 107, which are rotatable and mounted upright and respectively to the rear of the ultraviolet lamp 105.

Each of the pair of reflectors 107a, 107b is fixed in position such that one side is secured to a rotation axis and the other side is moved by a driving force from the sterilization angle adjusting unit 111 along a path formed by corresponding guides 420a, 420b, to adjust the sterilization angle θ (see FIG. 4).

The rotation axes of each of the pair of reflectors 107a, 107b are located adjacent to each other at the rear of the ultraviolet lamp 105, and the pair of reflectors 107a, 10b are positioned to form a V shape, —shape, \hat{ }shape or I shape at the rear of the ultraviolet lamp 105 depending on a size of the sterilization angle θ.

The sterilization angle θ refers to an angle formed by the pair of reflectors 107a, 107b in a direction of positioning the ultraviolet lamp 105 inwardly to form the sterilization region (see FIG. 5) in which ultraviolet rays generated by the ultraviolet lamp 105 directly propagates or are reflected by the reflectors. As will be described later, the sterilization angle serves as a basis for determining a conditional time constant value P, which is a factor considered when calculating an operation time t of the ultraviolet sterilization device 100.

As illustrated in FIG. 3, depending on the size of the sterilization angle θ formed by each of the pair of reflectors 107a, 107b reflecting ultraviolet ray generated by the ultraviolet lamp 105 in a rotated position, the ultraviolet sterilization device 100 may be operated for sterilization processing in a narrow-range mode, a mid-range mode, or a wide-range mode.

The narrow-range mode represents a sterilization operation mode in which the pair of reflectors 107a, 107b are rotated to form an acute sterilization angle. Since the sterilization angle is formed at a relatively small angle in the narrow-range mode, the sterilization region is formed relatively narrowly, but even the ultraviolet rays reflected by the pair of reflectors 107a, 107b are concentrated in the narrow area, making the sterilization power relatively strong.

In comparison, the mid-range mode is a sterilization operation mode at a sterilization angle of 180 degrees, and the wide-range mode is a sterilization operation mode at an obtuse sterilization angle. In the wide-area mode, the sterilization angle is formed at a relatively large angle, so the sterilization region is formed relatively wide, and the ultraviolet rays generated from the ultraviolet lamp 105 spread over a wide area, so the sterilization power is relatively reduced.

The sensor unit 109 may include one or more distance detection sensors configured for generating a distance measurement value for a distance to the target surface and providing it to the control unit 115.

While FIG. 8 illustrates a case where a total of four distance detection sensors are spaced 90 degrees apart on the body 210 of the ultraviolet sterilization device 100, but it will be appreciated that the number and spacing of the distance detection sensors mounted on the body 210 are not limited thereto.

However, the ultraviolet sterilization device 100 may be limited to having at least one distance detection sensor mounted on a front region of the body 210 (see FIGS. 2 and 4). The front region of the ultraviolet sterilization device 100 is in a direction in which the distance detection sensor S1 is mounted in FIG. 5, and refers to a region of the body 210 where ultraviolet rays generated from the ultraviolet lamp 105 is emitted to the outside even when the pair of reflectors 107a, 107b are rotated to minimize the sterilization angle θ.

In order to form a designated sterilization angle, the sterilization angle adjustment unit 111 is configured for providing a driving force that causes each of the pair of reflectors 107a, 107b disposed upright at the rear of the ultraviolet lamp 105 to move and rotate along corresponding guides 420a, 420b.

As illustrated in FIGS. 2, 3, and 7, the sterilization angle adjusting unit 111 may include an adjuster connected to each of the pair of reflectors 107a, 107b and configured for moving along the path disposed in a horizontal direction in front of the ultraviolet lamp 105 by control of the control unit 115.

When the adjuster moves along the path toward or away from the ultraviolet lamp 105, the pair of reflectors 107a, 107b connected to the adjuster are rotated at a mirrored angle to spread or converge with each other, thereby adjusting the sterilization angle θ.

Since the pair of reflectors 107a, 107b are commonly secured to the adjuster in the sterilization angle adjusting unit 111 illustrated in FIG. 2 and the like, the pair of reflectors 107a, 107b can rotate at an angle that mirrors each other by a move of the adjuster along the path.

In contrast, as illustrated in FIG. 4, in order to control the rotation of the first reflector 107a and the second reflector 107b at different angles, a first adjuster 111 a and a second adjuster 111b may be provided to correspond to each of the first reflector 107a and the second reflector 107b.

That is, by forming the path for the first adjuster 111a and the second adjuster 111b to move on an inner circumferential surface of the body 210 and each of the first adjuster 111a and the second adjuster 111b is secured to the corresponding reflector by connection means 410a, 410b, when each adjuster moves along the path, the corresponding reflector secured by the connection means can be moved and rotated along the corresponding guide.

The wheel unit 113 may include one or more wheels mounted on the body 210. The wheels unit 113 may include, for example, freely rotating wheels mounted to a lower portion of the body 210 to facilitate movement of the ultraviolet sterilization device 100, and wheels rotated by a drive motor to position the ultraviolet sterilization device 100 in response to a control at the control unit 115 for performing sterilization operations in accordance with the omni-directional option.

The control unit 115 is configured for controlling the operation of each component so that the ultraviolet sterilization device 100 performs the sterilization operation according to operation options corresponding to the user command input through the input unit 101.

Specifically, in order to perform the adaptive option or the omni-directional option, the control unit 115 determines the sterilization angle θ and the operation time t of the ultraviolet sterilization device 100 using the distance measurement value generated by the sensor unit 109 provided in the body 210.

To ensure that the distance to the target surface is not distorted by, for example, a person moving in front of the distance detection sensor, the control unit 115 may be configured to apply a distance measurement value that is maintained at the same distance for a predetermined period of time or longer.

In determining the sterilization angle using the distance measurement, the control unit 115 may be preset to control, for example, to form a sterilization angle of a predetermined obtuse angle if the distance measurement value generated by the distance detection sensor is below a first threshold (wide-range mode), to control to form a sterilization angle of 180 degrees if the distance measurement value is above the first threshold and below a second threshold (mid-range mode), and to control to form a sterilization angle of a predetermined acute angle if the distance measurement value is above the second threshold (narrow-range mode).

Of course, in proportion to the size of the distance measurement value, a specific sterilization angle may be preset to be selected within a range from a minimum sterilization angle (i.e., where the pair of reflectors 107a, 107b are rotated to be as close as possible to the sides of the ultraviolet lamp 105) to a maximum sterilization angle (i.e., where the pair of reflectors 107a, 107b are rotated farthest from the UV lamp 105).

This is for ensuring that if the distance to the target surface is short, a sufficient sterilizing effect can be expected even if ultraviolet ray, which has a relatively small sterilizing power, reaches the target surface, but if the distance to the target surface is relatively long, ultraviolet ray, which has a relatively large sterilizing power, can be expected to reach the target surface. In addition, even if a small sterilization angle is set when the distance to the target surface is long, the sterilization region is expanded due to the distance to the target surface, so that a large area can be sterilized at the position of the target surface.

Additionally, the control unit 115 may refer to the distance measurement value generated by the sensor unit 109 and determine the operation time t of the ultraviolet sterilization device 100 according to Equation 1.

$$\\ \begin{matrix}t = α \times \frac{{L}^{2}}{p} \times β & < Equation 1 >\end{matrix}$$

Where, α is a basic time constant value predetermined for the ultraviolet sterilization device 100, L is the distance measurement value generated by the distance detection sensor, p is a power rating of the ultraviolet sterilization device 100 or the ultraviolet lamp 105, and μ is a conditional time constant value predetermined corresponding to the size of the sterilization angle.

The size of the sterilization angle and the size of the conditional time constant value corresponding to the distance measurement value may be predetermined as a setting reference information and stored in a storage unit (not shown) such as if the distance measurement value is 100 cm, the sterilization angle θ is predetermined as 90 degrees and the conditional time constant value μ is predetermined as 0.25, if the distance measurement value is 75 cm, the sterilization angle θ is predetermined as 180 degrees and the conditional time constant value μ is predetermined as 0.5, and if the distance measurement value is 50 cm, the sterilization angle θ is predetermined as 360 degrees and the conditional time constant value μ is predetermined as 1.

Therefore, as illustrated in FIG. 5, using the distance measurement value generated by the sensor unit 109, the control unit 115 may control the operation of the sterilization angle adjusting unit 111 to rotate the pair of reflectors 107a, 107b, respectively, to form a sterilization angle according to the predetermined setting reference information, and may control the ultraviolet sterilization device 100 to perform the sterilization operation by lighting the ultraviolet lamp 105 for the operation time calculated by applying the conditional time constant value according to the predetermined setting reference information.

As described above, ultraviolet rays have a harmful effect on the human body, so there is a risk of using them in spaces where people frequently enter and exit, and attempts are being made to ensure safety by turning off the ultraviolet lamp when a sensor detects that a person is entering the sterilized space.

However, stopping the operation of the ultraviolet sterilization device due to the presence of people in a space where people frequently enter and exit causes aggravation of the pollution level in the space.

In contrast, the control unit 115 of the ultraviolet sterilization device 100 according to embodiment adjusts the sterilizing area variably when a moving object such as a person is expected to enter the sterilization region where ultraviolet sterilization is performed, the sterilization region is adjusted variably so that a protective operation for the moving object against ultraviolet rays is performed.

Specifically, the control unit 115 determines whether the distance measurement values generated by each of the plurality of distance detection sensors mounted on the body 210 of the ultraviolet sterilization device 100 change.

For example, assuming that a plurality of distance detection sensors is mounted along the circumferential surface of the body 210, if the distance measurement values generated by each of the distance detection sensors for the target surface remain unchanged, the control unit 115 can recognize that a moving object does not exist.

However, if the distance measurement values currently generated by one or more distance detection sensors indicate a shorter distance than the previous distance measurement values, the control unit 115 may recognize that a person 610, a moving object, is located at a forward position corresponding to the corresponding distance detection sensor.

As illustrated in (a) of FIG. 6, if the front position of the distance detection sensor is outside of the sterilization region where ultraviolet ray is irradiated, the control unit 115 may maintain the current sterilization angle and keep the ultraviolet lamp 105 turned on to ensure that the sterilization operation is maintained for the current sterilization region.

The control unit 115 may recognize the direction and speed at which the distance measurement values are changing by referring to, for example, which distance measurement values generated by each of the distance detection sensors are detected for which distance detection sensor in sequence, thereby recognizing the direction of move and the speed of move of the person 610.

As illustrated in (b) of FIG. 6, when the control unit 115 recognizes that the direction of move of the person 610 is toward the currently set sterilization region, the control unit 115 takes into account the speed of move of the person 610 (i.e., the speed at which changes in distance measurements are detected between adjacent distance sensors) to control the operation of the sterilization angle adjusting portion 111 to rotationally operate the corresponding reflectors so that the sterilization zone narrows in the opposite direction of the person 610 so that the person 610 does not enter the sterilization zone (see (c) of FIG. 6).

Thereafter, as illustrated in (c) and (d) of FIG. 6, when the control unit 115 recognizes that the person 610 moves again in the opposite direction of the adjusted sterilization region, the control unit 115 may control the corresponding reflector to rotate so that the sterilization region is again enlarged by the initially set sterilization region.

However, if the pair of reflectors 107a, 107b are positioned to form the minimum sterilization angle (the pair of reflectors 107a, 107b are rotated to be as close as possible) and the person 610 is still moving toward the sterilization region, the control unit 115 may control the ultraviolet lamp 105 to turn off.

In addition, if the front position of the distance detection sensor that first recognizes the presence of the person 610 corresponds to the sterilization region where ultraviolet ray is irradiated, the control unit 115 may control that the ultraviolet lamp 105 is turned off, or may control that a predetermined protective operation is performed, such as causing the sterilization angle adjusting unit 111 to rapidly rotate the reflector to adjust the sterilization region, as described above.

In addition to rotation of the reflector as a whole, the adjustment of the sterilization region for protective operation against moving objects may also be implemented by adopting a rotational structure of a portion of the reflector, as exemplified in FIG. 7, or by positioning a pair of shielding members 720a, 720b adjacent to the inner circumferential surface of the body 210.

As shown in (a) of FIG. 7, each of the first and second reflectors 107a, 107b is divided into the first sub-reflectors 107a-1, 107b-1 disposed at the center of rotation axes and the second sub-reflectors 107a-2, 107b-2 located outwardly, and the first sub-reflectors 107a-1, 107b-1 and the second sub-reflectors 107a-2, 107b-2 may be configured to be coupled to each other via rotating units 710a, 710b that rotate the second sub-reflectors 107a-2, 107b-2 relative to the first sub-reflectors 107a-1, 107b-1.

In this case, when it is desired to reduce or expand area of the sterilization region, the control unit 115 controls the rotating units 710a and 710b to cause the second sub-reflectors 107a-2, 107b-2 to be rotated relative to the first sub-reflectors 107a-1, 107b-1. In this case, it is advantageous that, by rotating only the relatively small second sub-reflectors 107a-2, 107b-2, the area of the sterilization region can be reduced or expanded relatively quickly compared to the case of rotating the entire reflector described above.

In addition, as shown in (b) of FIG. 7, the pair of shielding members 720a, 720b having a structure that folds and unfolds, for example, like a fan, can be disposed adjacent to the inner circumferential surface of the body 210. When it is desired to reduce or restore the area of the sterilization region, the control unit 115 may control the operation of corresponding driving means (not shown) to cause the pair of shielding members 720a, 720b to unfold or fold outwardly of the pair of reflectors 107a, 107b.

And, as described above, the ultraviolet sterilization device 100 according to this embodiment can operate with various operation options such as the manual option, the adaptive option, and the omni-directional option.

The manual option performs the sterilization operation according to selection information specified by the user to operate for a certain period of time in any one of wide-range mode, mid-range mode, and narrow-range mode.

In contrast, the adaptive option and the omni-directional option are operation options in which the ultraviolet sterilization device 100 performs the sterilization operation by calculating the sterilization angle and operation time corresponding to the distance measurement value to the target surface adaptively to the surrounding environment.

However, the difference is that the adaptive option performs the sterilization operation while maintaining the position in which the ultraviolet sterilization device 100 is placed, while the omni-directional option performs the sterilization operation while rotating 360 degrees in the position in which the ultraviolet sterilization device 100 is placed, so that the sterilization operation according to the adaptive option is performed in all directions.

When a plurality of distance detection sensors are mounted on the outer circumferential surface of the body 210 of the ultraviolet sterilization device 100, the distance measurement values to the target surface are generated by each distance detection sensor, and then the ultraviolet sterilization device 100 perform the sterilization operation according to the sterilization angle and the operation time corresponding to the distance to the target surface in each direction, so that the sterilization processing in all directions can be completed.

Of course, even when one distance detection sensor is mounted in the front region of the body 210, the control unit 115 of the ultraviolet sterilization device 100 may repeat performing the sterilization operation with the sterilization angle and the operation time corresponding to the distance measurement value to the target surface generated in the state of being postured to face the first direction, controlling the operation of the wheel unit 113 to rotate by a predetermined angle, and performing the sterilization operation with the sterilization angle and the operation time corresponding to the distance measurement value to the target surface generated in the state of being postured to face the second direction, so that the sterilization processing for the entire peripheral direction of the ultraviolet sterilization device 100 is completed.

As shown in FIG. 8, if the distance measurement values from each lateral direction of the ultraviolet sterilization device 100 to the target surface are different, it will be appreciated that the sterilization angle and the operation time in each direction will be set differently to correspondingly.

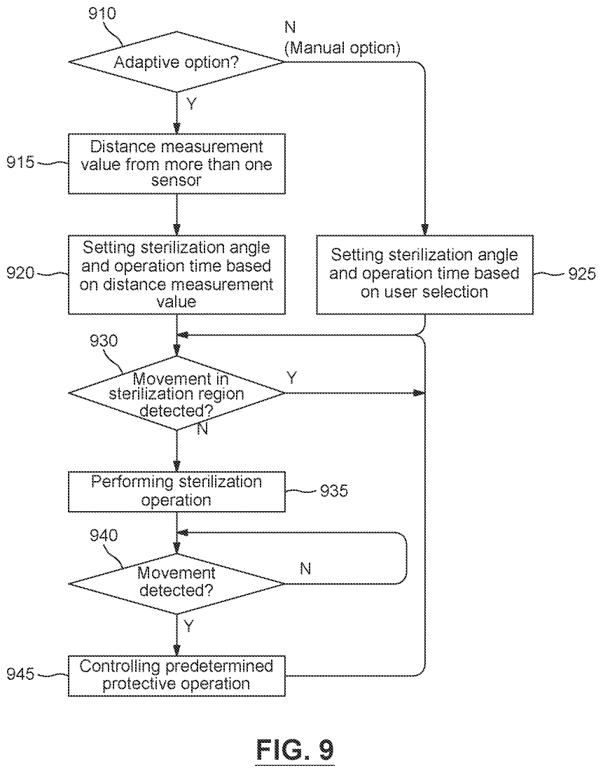

FIG. 9 is a flowchart showing a method for controlling the operation of the ultraviolet sterilization device according to one embodiment of the present invention.

Referring to FIG. 9, in step 910, the control unit 115 determines whether the adaptive option is selected as the user command for operating the ultraviolet sterilization device 100.

If the adaptive option is selected, in step 915, the control unit 115 receives the distance measurement value to the target surface generated by the distance detection sensor mounted on the body 210. The distance detection sensor that generates the distance measurement value may include at least a distance detection sensor mounted on the front region of the body 210.

In step 920, the control unit 115 sets the sterilization angle θ according to predetermined setting reference information based on the input distance measurement value and applies the conditional time constant value μ corresponding to the sterilization angle to set the operation time t of the ultraviolet sterilization device 100 (see Equation 1 above).

To ensure that the distance to the target surface is not distorted by, for example, a person moving in front of the distance detection sensor, the control unit 115 may be configured to recognize as a distance measurement value to the target surface a distance that is recognized by the distance measurement value to be maintained for a predetermined period of time or longer.

However, if it is determined in step 910 that the user command is for the manual option, in step 920, the control unit 115 sets the sterilization angle and the operation time to correspond to the user's selection.

In step 930, the control unit 115 determines whether the moving object such as a person is detected within the sterilization region to be formed to correspond to the set sterilization angle θ. Whether the moving object exists within the sterilization region to be formed may be determined by referring to whether the distance measurement value generated by the corresponding distance detection sensor changes.

If the moving object is detected within the sterilization region to be formed, the device waits until the moving object within the sterilization region disappears to prevent danger caused by ultraviolet ray.

However, if no moving object is detected in the sterilization region to be formed, in step 935, the control unit 115 controls the pair of reflectors 107a, 107b to rotate so that the set sterilization angle is formed, and the ultraviolet lamp 105 to be turned on to perform sterilization operation on the sterilization region for the set operation time.

While the sterilization operation is being performed before the set operation time expires, the control unit 115 determines whether a moving object exists in the space where the ultraviolet sterilization device 100 is located by referring to the distance measurement value generated by the distance detection sensor (step 940).

A plurality of distance detection sensors may be mounted on the circumferential surface of the body 210 of the ultraviolet sterilization device 100 so that the control unit 115 can recognize the presence of a moving object in the circumferential direction of the ultraviolet sterilization device 100 by referencing a change in the distance measurement values.

If there is no moving object, the control unit 115 controls the sterilization operation to be maintained.

However, if a moving object exists, in step 945, the control unit 115 controls the operation of each component so that a preset protective operation is performed corresponding to the position of the moving object.

Here, the preset protective operation may be, for example, controlling the sterilization region to be reduced or controlling the ultraviolet lamp 105 to be turned off.

In particular, the control unit 115 can recognize the direction of move and the speed of move of the moving object by referring to the mounting positions of each of the distance detection sensors at which the generated distance measurement value changes. If the moving object moves toward the currently set sterilization region, the corresponding reflector can be rotated and controlled to reduce the area of the sterilization region in a way that prevents the moving object from entering the sterilization region.

Of course, if the moving object is recognized as moving in the opposite direction of the sterilization region, the control unit 115 may control the rotation of the corresponding reflector to restore the area of the sterilization region, and if the reflector is rotated to achieve the minimum sterilization angle and the moving object is still moving toward the sterilization region, the ultraviolet lamp 105 can be controlled to turn off.

Based on the determination of step 910 described above, if the user selects the omni-directional option, the control unit 115 may repeat, until 360 degrees rotation is achieved, performing step 915 to step 945 for the first direction, controlling the wheel unit 113 to cause the ultraviolet sterilization device 100 to rotate by a predetermined angle, and performing step 915 to step 945 for the second direction, so that sterilization processing is performed for the entire circumferential direction of the ultraviolet sterilization device 100.

Of course, if the ultraviolet sterilization device 100 is designated to operate in the omni-directional option, it is appreciated that the rotation angle of the ultraviolet sterilization device 100 is not limited to 360 degrees, and may be set to 720 degrees, or the like, or may be set to operate in a continuous rotation until a stop command is entered by a user.

It will be appreciated that the above-described method of controlling the operation of the ultraviolet sterilization device may also be implemented as a software program, application, or the like embedded in a digital processing device and performed as an automated procedure in a time-serial sequence. The codes and code segments constituting the programs and the like may be readily made by a computer programmer skilled in the art. Further, the program may be stored on computer readable media, read and executed by a computer to implement the method.

Although the present invention has been described above with reference to embodiments, it will be understood by one of ordinary skill in the art that various modifications and changes can be made to the present invention without departing from the spirits and scope of the present invention as recited in the scope of the following patent claims.

What is claimed is:

1. An ultraviolet sterilization device comprising:
an ultraviolet lamp, mounted on an interior of a body, configured for generating ultraviolet rays;
a pair of reflectors, having surfaces reflecting the ultraviolet rays, being rotatable around respective rotation axes located adjacent to each other at the rear of the ultraviolet lamp;
a sterilization angle adjusting unit, configured for rotating each reflector around its corresponding rotation axis;
a sensor unit, having a distance detection sensor that generates a distance measurement value related to the distance to a target surface; and
a control unit, configured for controlling an operation of the sterilization angle adjusting unit to rotate each of the pair of reflectors so that a predetermined sterilization angle corresponding to the distance measurement value is formed,
wherein the sterilization angle is an angle formed between the pair of reflectors in a direction opposite the ultraviolet lamp made and a sterilization region through which the ultraviolet rays generated by the ultraviolet lamp pass is limited by the pair of reflectors that are rotated and positioned to form the sterilization angle.

2. The ultraviolet sterilization device of claim 1, wherein the control unit uses Equation 1 to calculate an operation time t for generating the ultraviolet rays for a sterilization operation,
$$\\ \begin{matrix}t = α \times \frac{{L}^{2}}{p} \times β & < Equation 1 >\end{matrix}$$
where α is a predetermined basic time constant value, L is the distance measurement value generated by the distance detection sensor, p is a power rating of the ultraviolet sterilization device or the ultraviolet lamp, and μ is a predetermined conditional time constant value corresponding to a size of the sterilization angle.

3. The ultraviolet sterilization device of claim 2, wherein the control unit controls the sterilization angle adjusting unit such that the sterilization angle of a relatively small angle is formed as the distance measurement value becomes relatively distant,
wherein the relatively small conditional time constant value is preset to be determined for the sterilization angle of the relatively small angle.

4. The ultraviolet sterilization device of claim 2 further comprising a wheel mounted on a lower portion of the body and rotated by a drive motor,
wherein in order to ensure that the sterilization operation is performed for the entire lateral direction of the ultraviolet sterilization device, the control unit repeats a series of steps of controlling the drive motor to rotate the ultraviolet sterilization device by a predetermined angle with a vertical rotation axis as a rotation center to face the second direction after completing the sterilization operation for the operation time calculated while the ultraviolet sterilization device is postured to face a first direction, and calculating the sterilization angle and the operation time for performing the sterilization operation while postured to face a second direction.

5. The ultraviolet sterilization device of claim 1, wherein the sensor unit comprises a plurality of distance detection sensors each mounted at a predetermined location on a circumferential surface of the body,
wherein the control unit refers to whether the distance measurement values generated by the plurality of distance detection sensor change to recognize a presence of a moving object, a direction of move of the moving object and a speed of move of the moving object.

6. The ultraviolet sterilization device of claim 5, wherein when recognizing that the moving object is approaching the sterilization region, the control unit controls the sterilization angle adjusting unit to rotate corresponding reflector in order to reduce an area of a currently set sterilization region in such a way that the entry of the moving object into the sterilization region is prevented.

7. The ultraviolet sterilization device of claim 6, wherein the control unit controls the ultraviolet lamp to turn off when the moving object is recognized as approaching toward the sterilization region with the reflector rotated to form a predetermined minimum sterilization angle.

8. The ultraviolet sterilization device of claim 5, wherein each of the pair of reflectors comprises a first sub-reflector coupled to the rotation axis, and a second sub-reflector coupled to the first sub-reflector by means of a rotating unit,
wherein when the moving object is recognized as approaching towards the sterilization region, the control unit controls the rotating unit to rotate the corresponding second sub-reflector in order to reduce an area of a currently set sterilization region in such a way that the entry of the moving object into the sterilization region is prevented.

9. The ultraviolet sterilization device of claim 5 further comprising a shielding member having a structure that folds and unfolds being disposed adjacent to the inner circumferential surface of the body,
wherein when the moving object is recognized as approaching towards the sterilization region, the control unit controls the shielding member to unfold in order to reduce an area of a currently set sterilization region in such a way that the entry of the moving object into the sterilization region is prevented.

10. The ultraviolet sterilization device of claim 1, wherein a surface of the pair of reflectors may be coated with magnesium oxide of a predetermined thickness.

11. The ultraviolet sterilization device of claim 1, wherein the control unit determines the sterilization angle to be formed based on a distance measurement that is maintained for a predetermined period of time or longer.

12. A computer program for performing al method of controlling operation of a ultraviolet sterilization device stored on a computer readable medium, wherein the computer program causes a computer to execute following steps:
designating a sterilization angle according to a predetermined setting reference information using a distance measurement value of the distance from the target surface, and determining an operation time t using Equation 1;
controlling a sterilization angle adjusting unit so that each of a pair of reflectors is rotated to form the sterilization angle; and
turning an ultraviolet lamp during the determined operation time,
wherein the sterilization angle is an angle formed between the pair of reflectors in a direction opposite the ultraviolet lamp made and a sterilization region through which the ultraviolet rays generated by the ultraviolet lamp pass is limited by the pair of reflectors that are rotated and positioned to form the sterilization angle,
$$\\ \begin{matrix}t = α \times \frac{{L}^{2}}{p} \times β & < Equation 1 >\end{matrix}$$
where α is a predetermined basic time constant value, L is the distance measurement value generated by the distance detection sensor, p is a power rating of the ultraviolet sterilization device or the ultraviolet lamp, and μ is a predetermined conditional time constant value corresponding to a size of the sterilization angle.
